(19) United States
(12) Patent Application Publication
(10) Pub. No.: US 2007/0015815 A1
(43) Pub. Date: Jan. 18, 2007

(54) Hydrazide substrate shuts down protein biosynthesis capability in cells that host a metastatic or malignant disease mechanism

(76) Inventor: Reuben E. Deloach, Fort Worth, TX (US)

Correspondence Address: REUBEN E. DELOACH, 3916 LINKMEADOW DR., FORT WORTH, TX, 76008, US

(21) Appl. No.: 11/524,559

(22) Filed: Sep. 21, 2006

Related U.S. Application Data
(63) Continuation-in-part of application No. 10/813,384, filed on Mar. 30, 2004, now abandoned.
(60) Provisional application No. 60/459,694, filed on Apr. 2, 2003.

(51) Int. Cl. A61K 31/405; C07D 209/44

(52) U.S. Cl. 514/419; 548/471

(57) ABSTRACT

The MAOI hydrazide substrate is targeted by protease cleavage in cells that host cancer, viral infections, or other malignant diseases because the hydrazide substrate (R′NHNHCOR″) simulates the peptide bonds (R′NHCOR″) that are innately targeted by protease cleavage. However cleavage of the hydrazide substrate forms a hydrazine radical which bonds to the protease enzyme to provide an irreversible substrate action that shuts down cell protein biosynthesis thereafter. Such process renders the malignant host cells sterile, static, and doomed to apoptosis where a disease free replacements cell can then be provided. The MAOI hydrazide drug also provides a new antibiotic purpose by rendering cells innate to infectious organisms sterile, static, and therefore harmless.

### CROSS REFERENCE TO RELATED APPLICATIONS

This is a continuation in part of application Ser. No. 10/813,384, filed Mar. 30, 2004, now abandoned. This application claims benefits of Provisional Application 60/459,694, filed Apr. 2, 2003.

### STATEMENT REGARDING FEDERALLY SPONSORED RESEARCH

(Not Applicable)

### BACKGROUND OF THE INVENTION

Field of Endeavor for this Invention

This invention pertains to the mono amine oxidase inhibitor (MAOI) hydrazide drugs that shuts down cells with a high level of protein biosynthesis activity that prevents oxidase proteins which provided an antidepressant drug purpose, but herewith such drugs provide a new purpose to target and shutdown cells with a high level of protein biosynthesis activity that is indicative of malignant disease activity as cancer, viral infections, or other metastatic disease activity. A related antibiotic purpose is also provided that shuts down protein biosynthesis in cells innate to infectious organisms thus rendering the organisms unable to reproduce, or provide toxic or dangerous protein products.

Introduction Overview

This invention began as a matter of curiosity about Malazide, or maleic acid hydrazide, a prior art invention that benignly shutdown cell division and growth of plants without harming the plants (Merck Index, 11th ed., p.5587). Such phenomena provoked the question whether such mechanism could be adapted for human use to shutdown cell division and growth of cancer. The applicant observed that the hydrazide molecule (R′NHNHCOR″) contains the amide pattern of a protein peptide bond (R′NHCOR″) that would provide a target for protease cleavage action in cells where protein biosynthesis was ongoing. If the cells were malignant then the large amount of protein biosynthesis ongoing will likely have depleted most protein substrate supplies in such cells and therefore the hydrazide substrate would be without substrate competition and be more quickly targeted by protease cleavage. However protease cleavage action that divides the hydrazide substrate would theoretically produce a hydrazine radical that would immediately bond to the protease enzyme molecule thus rendering the protease enzyme dysfunctional. And because a dysfunctional protease enzyme cannot support protein biosynthesis to make its own needed replacement an irreversible substrate action is created where the cell is rendered sterile and static, and doomed to eventual apoptosis, and can then be replaced by a new healthy cell. Because hydrazide is biologically inactive until protease enzyme cleavage releases the hydrazine radical which then reacts to inhibit protease, such process constitutes a prodrug irreversible substrate protease inhibitor for cells under heavy protein biosynthesis demands. The prodrug mechanism is analogous to an electrical fuse that interrupts electrical flow when the level of current exceeds a safe level. The hydrazide substrate is somewhat a biological equivalent of such fuse which will interrupt protein biosynthesis in any cell when protein biosynthesis demand is high which indicates a malignant or metastatic type condition.

In effort to test the applicant's hypothesis Iproniazid, was used to supply a hydrazide substrate to pregnant mice which served as animal models somewhat representative of cancer because of the rapid cell division and growth of fetal cells. And as was predicted the benign hydrazide shutdown of fetal cell division and growth was evidenced by the birth of all healthy mice that matured normally to provide midget size adults. A further search of hydrazide prior art also revealed an antiviral hydrazide, Marboran, which also evidenced the irreversible substrate process used to shutdown protein biosynthesis in cells that hosted smallpox, polio, and other viruses that also made heavy demands for viral metastatic protein which illustrated an efficacious, rapid acting total spectrum antiviral drug based on the same hydrazide mechanism (Cutting's Handbook of Pharm. 6th ed., p.125). Several other prior art hydrazides were also found that likewise evidenced the irreversible substrate mechanism as addressed in the prior art section. Such overwhelming evidence of a benign, rapid acting, and powerful anticancer and antiviral drug mechanism based on an existing antidepressant pharmaceutical hydrazide product that has a 50 years history of safe use prepared the way for human testing where the MAOI hydrazide drug, Iproniazid, was then used with phenomenal success to shutdown the HIV/AIDS virus, viral conjunctivitis, influenza virus, malignant melanoma, basal cell carcinoma, Kaposi's Sarcoma, and bacterial gastroenteritis.

General Information and Description

Hydrazides were first used by Fox in 1951 as a tuberculosis agent provided under the name Isoniazid, that was modified with isopropyl to provide Iproniazid that unexpectedly raised the mood in tuberculosis patients which was the beginning of the MAOI hydrazide antidepressant class of drugs. The antidepressant hydrazide drug mechanism had remained a mystery although some theories pertaining to aspects of the drug had been postulated. For example it was theorized that hydrazide inhibits the release of norepinephrine from storage sites, Axelrod, J. Pharmacol. Exp. Ther., 134:325, 1961; that it stimulates receptors directly (Quastel); that it interferes with penetration of pressor amines to intracellular sites (Koelle); that DOPA decarboxylase is inhibited; and that a possible irreversible substrate shutdown of oxidase increases norepinephrine, Spector (J. Pharmacol. Exp. Ther., 128:15,1960).

The prevailing theory was that an irreversible hydrazide substrate penetrates the mono amine oxidase (MAO) site of the brain, and therein binds with the oxidase enzyme to negate its enzyme action where neural stimulants as norepinephrine then accumulate to provide the antidepressant effects (Cutting's Handbook of Pharm. 6th ed., p. 628, 1979). Such theory is somewhat stated in the hydrazide antidepressant drug label as a “mono amine oxidase inhibitor,” which would seemingly indicate that the hydrazide drug mechanism is as an inhibitor of oxidase enzyme action which is not correct. The absence of oxidase is only a manifestation of protein biosynthesis shutdown caused by true then undiscovered hydrazide mechanism. As such the applicant became involved in such quest to determine the biological mechanism of hydrazides which would help explain the phenomena of hydrazides as illustrated by Malazide that had properties that would benignly shutdown cell division and growth which seemed extraordinary and ideally suited as a cancer drug type mechanism and as such needed to be determined.

A search of the literature was successful to reveal prior art uses of hydrazides that all indicated a protein inhibiting connection. For example (1) Schoene and Hoffman in 1949, first used Malazide, the simple molecule with no functional groups other than the hydrazide that benignly shutdown cell division and growth of tobacco plants without harming the plants. No mechanism is suggested by the literature but obviously it is a hydrazide mechanism that inhibits protein. (2) Fox in 1951, provided Isoniazid, a hydrazide that provided chemotherapeutic action for tuberculosis use. The literature proposed that the hydrazide inhibited protein synthesis in the cell envelope. (3) Zeller in 1952, provided Iproniazid for antidepressant use where the literature proposes that an irreversible substrate mechanism prevents proteins as oxidase. (4) Thompson in 1953, discovered Marboran, a hydrazide that had total spectrum antiviral use that shutdown smallpox, polio virus, and other viruses tested. The literature proposes that a protein biosynthesis dysfunction prevented protein incorporation into the virus particle. All such hydrazide uses had an obvious protein controlling means responsible for the unique hydrazide properties illustrated.

The applicant had also taken notice of the similarity of the hydrazide molecule which contains an amide group, like that of peptide substrate, where the amide group also exists in the peptide bonds of the protein substrate material. And because protease cleavage targets the amide pattern in the substrate material that provides the proteolysis of substrate supplies used for the protein biosynthesis process, then protease cleavage would also target the hydrazide substrate amide pattern. In such case the cleavage action would divide the hydrazide molecule into two parts where the hydrazine radical part would then attach to the protease enzyme molecule providing its dysfunctional shutdown. However because a dysfunctional protease enzyme cannot support protein biosynthesis as needed to make its own replacement, the hydrazide substrate protease inhibitor would constitute an irreversible substrate action that would shutdown all protein biosynthesis capability for the cell. Such cell will remain in a static and sterile cell state until apoptosis claims the cell. Then such static cell will be replaced with a new cell. In effect hydrazide is a protease inhibitor that provides an irreversible substrate shutdown which prevents protein biosynthesis thereafter which provides an explanation for the prior art examples.

Such irreversible substrate concept has been postulated as an oxidase enzyme inhibitor which the applicant agrees is correct in context that such is a protease inhibitor and not an oxidase inhibitor. Such concept provides a hypothesis that logically explains all hydrazide biological prior art phenomena and can predict the prodrug type irreversible substrate action of any simple or low molecular weight hydrazide molecule. The irreversible substrate type protease inhibitor idea is also in agreement with the antidepressant drug explanation and working model, and the Malazide explanation and working model, and was predicted and illustrated by the mouse fetus experiment, and additionally concurs with the Marboran antiviral working model and explanation. The irreversible substrate protease inhibitor has additionally been reduced to practice when Iproniazid was first used to test antiviral capability when the applicant was taken ill by influenza and a small dose of Iproniazid provided for the very rapid recovery that followed. And following a long period of time where no research was conducted due to family tragedies and related obstruction, the applicant returned to such work where the MAOI type hydrazide, Iproniazid, was then successfully used to target and shutdown protein biosynthesis in cells that hosted malignant disease mechanisms comprising various types of cancers and viral infections. In effect a malignant cell targeting and eradication mode of use for the MAOI hydrazide had been conceived, shown to be provided by the irreversible substrate protease inhibitor action which provides a highly predictable prodrug process that always shuts down cell protein biosynthesis in cells where protein biosynthesis demands are representative of levels which indicates a malignant or metastatic cell condition.

Current Protein Inhibitor Drugs used for Malignant Disease Purposes

A history o protein inhibiting drugs exist that have commonly been used in the treatment of malignant disease because protein provides metastasis of the disease mechanism from cell to cell, and protein is a cause of pain and damage whereby inhibiting malignant cell proteins inhibits disease metastasis and pain. For example one of the first drugs used for Parkinson's was Bromocriptine which is a protein inhibitor illustrated by fact it also inhibits the natural growth hormone protein. And amantadine is a somewhat reversible substrate protein inhibitor that has also been used as an antiviral drug. Carbidopa is listed as a hydrazine (H2NNH2) drug which is a metabolic precursor for the hydrazide active drug principal which then becomes a powerful protein inhibitor method that targets only cells with high levels of ongoing protein biosynthesis as the present invention provides but only about half of the hydrazine drug administered is metabolized to the useable hydrazide form where the other half remains as a toxic hydrazine affront to the patient. Cancer likewise makes use of several additional hydrazine drugs which even though toxic, such drugs have remedial potential where Procarbazine is an anti-neoplastic drug derived from methyl-hydrazine. And hydrazine sulfate has “Investigational New Drug” status and is approved for cancer use abroad. The principal drugs making headway in the treatment for multiple myeloma are also protein inhibiting drugs illustrated by Thalidomide, Bortezomib, Trisenox, and a growing list of additional protein inhibitors. And a long list of protease inhibitors are used for HIV/AIDS which cannot shutdown the virus and also has serious side effects but nevertheless inhibits protein biosynthesis somewhat which inhibits the virus.

The hydrazine drug invention also provides such advanced mechanism as the present invention provides but only after the hydrazine is converted by metabolic action to provide the hydrazide product as the present invention provides without the metabolism step. As such hydrazines do provide needed improvement over existing protein inhibiting drugs as used for malignant disease purposes. Examples of such drugs provided by hydrazine are Carbidopa, Procarbazine, methylhydrazine, and hydrazine sulfate. Such hydrazine drugs nevertheless must be transformed to the hydrazide metabolite necessary to provide a viable hydrazide protein inhibitor molecule. Such hydrazide metabolite molecules then have the hydrazide irreversible substrate prodrug capability to be targeted by protease cleavage by cells that host the viral infections, cancer, or other malignant disease or conditions. However the hydrazine drugs are useful only to provide a metabolic precursor of the active drug principal which is an acetylhydrazide (H2NNHCOCH3″) metabolite, which has a hydrophilic nature that is quickly excreted and therefore requires higher dose levels and more frequent dosing than the MAOI hydrazide equivalent. And such metabolic change from hydrazine to hydrazide is slow and increases patient exposure to toxic hydrazine that was shown by Fox in 1951, that only about 50% of the hydrazine is metabolized and the rest remains as a toxic poison which is inherent to hydrazine (Cutting's Handbook of Pharm. 6th ed., p. 39-40, 1979).

And if the hydrazine drugs were replaced with the acetylhydrazide metabolite form the start, such would be an improvement that eliminates the hydrazine toxic effects, be faster acting, and would increase efficacy. However the acetylhydrazide is still ill suited as a pharmaceutical because it is hydrophilic and excreted quickly but can be improved upon further by substuring the hydrazine for the MAOI hydrazide type embodiment. Therefore improvement over the toxic hydrazine, and the short lived acetylhydrazide metabolite is provided by replacing the hydrazine drugs directly with the MAOI hydrazide drug embodiment which provides optimal performance in all respects that the hydrazine can provide, and which constitutes the most efficacious embodiment for targeting and eradicating cells that host a malignant and metastatic type disease activity. The MAOI hydrazide drugs have no hydrrazine toxic effects, such are fact acting, and requires a much lower dose level and therefore provides a much needed improvement over the currently used toxic hydrrazine drugs.

The MAOI hydrazide drug embodiment also provides optimal antiviral performance in all respects as it represents a time tested optimized efficacious irreversible substrate protease inhibitor prodrug embodiment having use for targeting and rendering sterile and static all cells providing persistent viral metastatic coat protein biosynthesis. Thompson in 1953, discovered the first hydrazide antiviral application when he discovered Marboran which had amazing total spectrum antiviral properties effective against all viruses and was tested against the most virulent viruses known at the time which were smallpox and the polio virus (Cutting's Handbook of Pharm. 6th ed., p. 125, 1979). However Thompson had made an apparent serendipitous discovery while using a toxic isatin thiosemicarbazone laboratory chemical reagent which unwittingly supplied the hydrazide prodrug substrate which is innate to the semicarbazone molecule that provided the antiviral prodrug mechanism. Unfortunately the toxic effects of the semicarbazone reagent chemical remained in the Marboran pharmaceutical product, and Marboran was discontinued. Such cytotoxicity problem is easily remedied in hindsight of the present understanding provided herein simply by replacing the toxic isatinthiosemicarbazone chemical that supplied the necessary hydrazide substrate with a nontoxic molecule with a hydrazide group such as that the MAOI hydrazide class of pharmaceuticals can. Iproniazid provides an example of such MAOI hydrazide pharmaceutical product that supplies a safe and non-cytotoxic hydrazide substrate which has a 50 year history of safe medical use abroad.

General Concepts Claimed by the Present Invention

The first concept claimed by this invention pertains to a new purpose provided by the MAOI hydrazide antidepressant drugs which provides a prodrug mechanism that targets and shuts down the protein biosynthesis capability in cells that host a malignant disease. This concept is based on the MAOI hydrazide irreversible substrate process that is likewise used to shutdown oxidase and other protein biosynthesis in CNS cells that provides the MAOI antidepressant mode of use, and such hydrazide substrate process likewise provides the means to target and shutdown cells that have ongoing protein biosynthesis loads as is indicative of cells that host a malignant disease as cancer, viral infections, or other metastatic disease mechanism. The malignant host cells shutdown by the process are then rendered sterile and static and doomed to apoptosis and replacement with a healthy new cell. Such concept has been reduced to practice as provisioned by Kirby, 40 USPQ 368.

A second concept claimed by this invention pertains to the improvement it provides over the Marboran antiviral drug which is an isatin thiosemicarbazone type hydrazide drug. Marboran contains the required hydrazide substrate group that provides an efficacious antiviral mechanism and use but also contains additional cytotoxic molecule structure in the semicarbazone that provides a reagent chemical use for separating ketone and aldehydes from solutions. As such the Marboran cytotoxicity problem is inherent to the toxic reagent molecule that is not akin to the hydrazide group that provides the antiviral mechanism and properties. As such the cytotoxic problem is remedied by replacing the toxic chemical reagent molecule with the cytotoxic free MAOI hydrazide type molecule which provides an important antiviral use discovered but cannot be uses except as provisioned here. As such a fast acting efficacious total viral spectrum capability using is made possible by using a hydrazide, and preferably by using the existing MAOI type pharmaceutical product. Such antiviral improvement has been reduced to practice as described herein.

A third concept claimed by this invention pertains to improvements it provides over the hydrazine class of drugs that provide essentially the same medical uses and purpose as the MAOI hydrazide drug provides as a malignant disease treatment drug. However the toxic hydrazine drug product is inactive as a protein inhibitor until converted by system metabolism action to provide the hydrazide prodrug principal. Such indirect method used to provide the active hydrazide drug principal exposes the patient to unnecessary delay and exposure to hydrazine toxic effects and diminished benefits otherwise made possible by hydrazide. As such this present invention eliminates the toxic, slow, and inefficient hydrazine metabolism conversion step by replacing such hydrazine with the MAOI hydrazide drug, which then provides a toxic free and very much more efficacious method for malignant disease use. The MAOI hydrazide is much more efficacious for malignant cell targeting, malignant cell protein shutdown, and malignant cell apoptotic removal of the host cell where such cells can then be replaced by new disease free cells. The MAOI hydrazide improvement over the hydrazine and the acetyhydrazide metabolite is illustrated by the very successful application of the MAOI hydrazide for malignant disease uses as described herein.

A fourth concept claimed by this invention pertains to a second new purpose provided by the MAOI hydrazide antidepressant drugs which herewith provides a new total spectrum antibiotic drug mechanism and treatment means. Such antibiotic mechanism is provided by the MAOI hydrazide that supplies the irreversible hydrazide substrate mechanism that is targeted by protease cleavage by the cells innate to infectious organisms which are providing persistent ongoing protein biosynthesis action as supplies cell division, growth, and toxic protein generation. Protease cleavage action invokes the irreversible substrate mechanism that renders the cells of such infectious organisms sterile, static, and harmless. As such the value of this new antibiotics method is not how quickly it kills infectious organism which may take a week or two for conventional antibiotics, but how quickly it can render a dangerous and infectious organism sterile, static, and harmless which may take less than an hour by comparison. It is the speed at which the MAOI hydrazide drug can shutdown cell protein biosynthesis that halt the venomous pain producing and dangerous protein products that makes the comparative difference between a benign acidophilus bacterium and the extremely dangerous anthrax bacteria. Such new antibiotic purpose has been reduced to practice as provisioned by Kirby, 40 USPQ 368, as described herein.

The Preferred Hydrazide Embodiment

The preferred hydrazide embodiment is exemplified by Iproniazid, Isocarboxazid, and Nialamide which are current pharmaceutical drugs used abroad for antidepressant purposes that have a long history of safe use without cytotoxic effects as is a common problem with many non-MAOI hydrazide molecule structures. However the MAOI hydrazides are cytotoxic free and are generally provided by molecules that have molecular weights between 150 and 300 that consist of a carboxyl group condensed with an alkyl hydrazine group where the alkyl portion may be extended further in a somewhat straight line manner. Such molecules are essentially inert and unreactive until their prodrug mechanism is invoked by protease cleavage that targets the hydrazide substrate. Such cleavage action then provides molecular bonding of the substrate to the protease enzyme that renders the protease enzyme system dysfunction which prevent cell protein biosynthesis thereafter. The MAOI drug Iproniazid, (Marsilid) as was used most by this applicant has a therapeutic dose of 50-150 mg daily as an antidepressant drug and has 1760 mg/kg lethal dose for mice as listed by Psychotropic Drugs and Related Compounds, Public Health Service Pub. No.1589 (1967).

The Hydrazide Biological Mechanism

The applicant became interested in hydrazides in the early 60's and recognized the validity of an irreversible substrate theory in a new context as a protease inhibitor drug rather than a oxidase inhibitors as somewhat was claimed. It is protease cleavage targeting of the hydrazide substrate that renders the protease enzyme dysfunctional such that it cannot support protein biosynthesis action thereafter. Also apparent to the applicant was that the hydrazide molecule group (R′NHNHCOR″) contains a molecular sequence representative of the amide group (R′NHCOR″) that provides peptide bonds in protein substrate supplies. That proteolysis process is provided by protease cleavage that innately targets the amide groups of such peptide substrates to supply the current ongoing protein biosynthesis activity. The protease cleavage targets the amide molecular sequence as exists in the hydrazide substrate molecule involves forced cleavage that generates a hydrazine radical that then bonds to the protease enzyme molecule that renders the protease system dysfunctional. And without a functional protease enzyme system all cell protein biosynthesis action ceases which renders the cell sterile and static without killing the cell. However apoptosis appears to claim the cell within a two week window where it is then replaced by a new healthy cell.

Potential Medical Uses for Hydrazide

Prior art inventors of the 1950's era made discoveries that were the unwitting result of hydrazide use as an irreversible substrate protease inhibitor that rendered malignant cell protease enzymes dysfunctional that provides medical uses illustrated by the following facts:
(a) that without a functioning protease enzyme a viral infected cell cannot provide protein biosynthesis of viral coat or metastatic protein which propagates the disease;
(b) that without a functioning protease enzyme a cancer cell cannot provide protein biosynthesis of peptide signals that induce cancerous cell division, or cancer metastatic products or provide growth;
(c) that without a functioning protease enzyme a malignant cell cannot produce toxic or aberrant protein product as exists for many malignant diseases or mutant cell conditions;
(d) that without a functioning protease enzyme active cells innate to infectious organisms are rendered sterile and static and cannot produce biosynthesis of peptides that signals or induces cell division, and as such the infectious organisms cannot reproduce, grow, or proliferate;
(e) that without a functioning protease enzyme the cells innate to infectious organisms cannot produce toxic or dangerous proteins, or pass on antibiotic resistance traits, or produce enzymes needed to negate or repair antibiotic caused damage. The following prior art provides working examples of the same hydrazide mechanism used by the present invention in context of medical uses.

Prior Art Hydrazide Halts Peptides for Plant Growth Inhibitor Purposes

Malazide or maleic acid hydrazide, (U.S. Rubber Co., U.S. Pat. No. 2,575,954,1951) was used by Schoene and Hoffman in 1949 as a plant growth inhibitor that was first used as a spray to stop “suckering” or new plant growth in tobacco farming. Such chemical spray was applied before harvest time probably to increase nicotine content. Similar uses later existed where maleic hydrazide was used to prevent biological changes as budding, ripening, and to retard spoilage of farm produce, and extensively used to prevent the sprouting of onions and potatoes held in storage. The biological mechanism is true to the irreversible substrate mechanism addressed above because the hydrazide substrate is targeted by plant cell protease cleavage action whenever a change is induced. The dysfunctional shutdown of protease then follows which renders such cells static and sterile and thus unable to provide protein biosynthesis necessary to produce the protein or peptide signals responsible for initiating changes as cell division, budding, ripening, and other natural processes. As such the hydrazide provides use to extend the shelf life of farm produce and to inhibit new growth of tobacco plants. Such hydrazide use has shown a safe history for for tobacco and grocery produce items used and consumed by the public.

Prior Art Hydrazide Halts Peptides for Tuberculostatic Purposes

Isoniazid or Isonicotinic acid hydrazide (U.S. Pat. No. 2,830,994,1958 to Distillers Co.), was used by Fox in 1952, as a tuberculostatic agent, (Cutting's Handbook of Pharm. 6th ed., p. 40, 1979). In effect Fox discovered that this chemical functioned as a tuberculostatic agent that exceeded all other substances screened. Isoniazid was not of an alkyl-hydrazide design that provides a prodrug mechanism like the preferred embodiment of the present invention, but had an exposed hydrazine terminal having a reactive and toxic hydrazine nature. This toxic effect is attributed to hydrazine reactivity because without an alkyl or other blocking group it condenses with chemical functional groups found in serum. It combines as a single molecule with ester, ketone, and amide groups and as such is transformed in vivo to various compounds. However the antimicrobial effect sought by Fox needed to be detrimental to the tuberculosis organism and to some extent that was provided by Isoniazid at the comparatively high dose levels required which was injurious to the infectious organism but also toxic to the patient.

The Isoniazid molecule was noted by Fox as 50% converted by acetylation to the metabolite that would have provided a useful hydrazide drug effect was it not for the drug's hydrophilic and cytotoxic nature, and the rapid excretion found in about half the patients treated with Isoniazid. The applicant believes that this metabolic pathway predominated because the reactive hydrazine terminal was exposed which reacted in vivo with plasma constituents because it did not have an alkyl or other substituent that would block its conjugation with the plasma components. Fox later tried the nontoxic alkyl form of Isoniazid which did block such metabolic pathway that negated the untoward toxic effects. That new product was provided under the name Iproniazid, but Iproniazid did not appear to kill the tuberculosis organism or seem to hasten its demise sufficiently which made Isoniazid his preferred choice as a tuberculostatic antibiotic agent. However Fox failed to discover that the infectious organism had been rendered sterile and static where replication was halted and the organism was essentially as inactive as if it were a dead organism which is illustrative of the antibiotic mechanism claimed by this invention.

Prior Art Hydrazide Halts Oxidase Protein for Antidepressant Purposes

Iproniazid or Isonicotinic acid 2-isopropylhydrazide, was researched in 1952, by Zeller that followed Fox's report that it raised the mood of patients given the drug during his testing on tuberculosis patients, (Cutting's Handbook of Pharm. 6th ed., p. 125; 1979).The research indicated that Iproniazid prevented amine oxidase and was therefore believed to be a mono oxidase enzyme inhibitor (MAOI) because it allowed biogenic amine stimulants to increase in the affected cells that explained the euphoria and positive spirits exhibited by the person receiving the drug. As a result Iproniazid was soon afterward produced as an antidepressant drug and the overwhelming therapeutic success motivated the development of Isocarboxazid, and Nialamide, which are still in use in some countries abroad.

The hydrazide type (MAOI) provides an irreversible hydrazide substrate type action. Such irreversible substrate action is provided when protease targets the hydrazide substrate that releases the hydrazine radical that bonds to the protease enzyme. The protease is rendered dysfunctional which shuts down protein biosynthesis action which inhibits oxidase protein production. Because the oxidase enzyme has a function to degrade biogenic amines, or neural stimulants, as norepinephrine, noradrenaline, and serotonin, the levels of such stimulants increases. The optimal levels are ordinarily provided by a biological regulation scheme that increases biosynthesis of oxidase enzymes as needed to oxidize or deactivate the increased levels of biogenic amines as such levels increases during CNS cell activity. The applicant believes that the biogenic amine degradation occurs only where the oxidase enzyme introduces oxygen to the alpha carbon of the biogenic amine stimulant molecules to form an amide function which has no stimulation effect, whereas the shutdown of oxidase biosynthesis prevents oxidase and hence oxidase degradation which allows biogenic stimulates levels to increase and remain at the peak level in active CNS cells as used in mental processes. The CNS cells in use then have increased stimulation but no means to provide cell protein biosynthesis activity and therefore remain electrically active as viable CNS cells until cell environment extremes trigger apoptosis days later when the cell is replaced with a new cell.

Prior Art Hydrazide Halts Viral Coat Proteins for Antiviral Purposes

In about 1953, Thompson discovered antiviral action provided by a thiosemicarbazone derivative, Methisazone or 1-Methylindole-2,3,-dione 3 thiosemicarbazone, (Cutting's Handbook of Pharm. 6th ed., p. 125). Such type of hydrazide was effective against smallpox, polio, and other viruses screened and the hydrazide reagent was provided under the name of Marboran. The applicant believes that Thompson had discovered the antiviral action existing in a hydrazide related laboratory reagent chemical which at relatively high dose levels was able to provide a hydrazide substrate action at levels sufficient to shutdown protease cleavage which ended the viral replication process. Unfortunately the hydrazide substrate supplied by the thiosemicarbazone molecule was poorly suited for pharmaceutical uses. As such the applicant believes that Thompson had made a serendipitous discovery related to a semicarbazone laboratory reagent he used to separate ketones and aldehydes from solution. Such could provide a false discovery as a laboratory technician might do as an attempt to extract viral antibodies from a patients urine which then would test beneficial against the virus, and would test beneficial against cancer or other malignant disease due to the hydrazide mechanism and not because of a supposed antibody. Additionally the very hydrophilic nature of the molecule used by Thompson required a comparatively large dose level to provide the hydrazide substrate equivalent of the protein inhibiting mechanism needed to halt viral protein biosynthesis. Such comparatively large dose also provided undue cytotoxic side effects. The literature proposes that this antiviral discovery worked because it caused a defect in protein incorporation. The applicant agrees with such explanation because the hydrazide shutdown of protease halts cell protein biosynthesis and hence provides an absence of protein for attachment of mRNA to the ribosome as cited.

Use of the Marboran antiviral agent was accompanied by nausea and vomiting due to cytotoxic effects. The dosage level required by the MAOI hydrazide to shutdown cell protease is illustrated by a comparison to Iproniazid, which is a dose level of 50 mg to 100 mg, whereas with the hydrophilic nature of the Marboran molecule used by Thompson at a dose level of 1500 mg to 3000 mg daily. The applicant believes that had Thompson discovered the hydrazide active principal based on protease cleavage action that made his antiviral molecule work he could have used a simple hydrazide substrate more easily targeted by protease cleavage and having better lipid solubility that would have yielded a very efficacious action at a very small comparative dose level. Based on the applicant's experience using Iproniazid, a rapid shutdown of any virus including HIV is possible at about a 100 mg dose level that has ended systemic viral infections including HIV, which should never take more than a few days and have no cytotoxic effects.

Prior Art Hydrazide Halts Degradation of Tissue Homogenates

Iproniazid is used for a third new purposes to preserve tissue homogenate. It is presently believed that Iproniazid inhibits oxidase which preserves the homogenate from change (See patent filings 20020136769 and 20030064988 which provides examples of such use). However hydrazide is not an oxidase inhibitor, which is another example of the mono amine oxidase inhibitor label being misleading for those who use such product. The fact is that the MAOI hydrazide is only an irreversible substrate protease enzyme inhibitor as previously discussed. It can only inhibit cell protease which in turn will prevent oxidase protein biosynthesis but it has no means to selectively inhibit or interface with the oxidase enzyme as a discreet inhibitor. Numerous types of enzymes exist that can alter a homogenate in numerous ways. Therefore the integrity of the homogenate is better protected by Iproniazid than thought. It is this ability to inhibit protease that provides the MAOI the capability to shutdown protein biosynthesis of all enzymes, toxic proteins, and protein needed for cell division and growth that provides the total spectrum antibiotic capability claimed by this invention. It is this total spectrum capability that likewise prevents live cells, bacterial, or other organisms from producing enzymes that might alter or petrify the homogenate.

Prior Art Hydrazine Halt Infectious Organisms, Tumors, Cancers, Parkinson's, etc.

Hydrazine (H2NNH2) supplies a hydrazide substrate that is targeted by protease cleavage but only after such hydrazine undergoes metabolic changes that provides the acetylhydrazide (H2NNHCOCH3″) metabolite. In 1952, Fox determined that such metabolic process was only about 50% accomplished where much of the hydrazine remains as a toxic substance having no medical value. Nevertheless the benefits derived by the hydrazide metabolite portion exceeds the disadvantages and is exemplified by the successful treatment of Cachexia and tumor inhibition using hydrazine as provided by the Syracuse Cancer Research Institute. And following recent clinical trials hydrazine sulfate, is likewise transformed in vivo to the hydrazide form, and hydrazine has been approved as an anticancer drug in Russia. Cefazolin is also a product widely used as an antibacterial hydrazine product, and Procarbazine provides antineoplastic use based on the monomethyl-hydrazine derivative which is also a metabolic precursor of the active hydrazide metabolite. Fluconazole likewise provides the hydrazine precursor of the more preferred hydrazide prodrug product which is the active principal that is being used to treat AIDS related fungal infections. Carbidopa is also reported as a treatment for Parkinson's disease and Furazolidone is likewise a hydrazide precursor effective against infectious organisms and is used in the veterinarian practice. Because such hydrazine drugs must be converted into hydrazide molecular form which then provides the malignant disease therapeutic purposes the prior art listed here actually illustrates the acetylhydrazide active principal being used for malignant disease treatment purposes. Such summary of hydrazine uses is provided by the Encyclopedia of Chemical Technology, 4^{th} ed., vol. 13, p. 597.

### BRIEF SUMMARY OF THE INVENTION

Summary of Prior Art Working Examples Used by the Invention

The prior art as listed above illustrates working examples of the hydrazide mechanism that provides malignant disease applications: (1) Malazide was introduced in 1949, as a plant growth inhibitor that shutdown cell protein biosynthesis for purposes to inhibit cell division and growth of plants which is a working example of the hydrazide mechanism used to shutdown cell division and growth of cancer, cells providing viral related proteins, and of cells innate to infectious organisms for antibiotic purposes. (2) Then in 1952, Iproniazid was used for tuberculostatic purposes but because it did not provide apoptotic evidence that it killed the bacilli the new type of antibiotic action that inhibits cell division, reproduction, toxic protein generation was not recognized and remained undiscovered until now. (3) Then about 1953, Iproniazid provided a process used to target and shutdown cells that had ongoing protein biosynthesis that included oxidase enzyme protein production that provides its antidepressant mode of use, which herewith is used to target and shutdown cell protein biosynthesis capability for cells that host a malignant or metastatic protein activity. (4) And about 1953, Marboran used hydrazide to target and shutdown cells that host smallpox, polio, and other viral infections which terminated all viral activity quickly that illustrates a total spectrum antiviral mechanism which is better provided by the MAOI hydrazide molecule which is without the cytotoxic problems of Marboran. (5) Then some time later Aldrich Chemical Company began supplying Iproniazid as a preservative for tissue homogenates which renders live cells, bacteria, and microorganisms sterile and static that otherwise would metabolize, putrefy, and alter the integrity of the homogenate which is an in vitro working example of the hydrazide antibiotic mechanism that renders infectious organisms sterile and static, and unable to produce toxic or damaging protein products as claimed by this invention.

The MAOI hydrazide provides the same uses and mechanism as hydrazine (H2NNH2) drugs provide after metabolic conversion to the hydrazide metabolite. Therefore the hydrazide and hydrazine have the same medical uses only that the hydrazine drug is toxic, slow to metabolize to the active hydrazide prodrug active principal, and is much less efficacious than the hydrazide for malignant disease treatment. However hydrazines are nevertheless used against cancer, Parkinson's disease, and infectious organism uses. Therefore the medical uses provided by hydrazine is in effect working examples of medical uses actually provided by the hydrazide metabolite that can be improved upon by substituting the hydrazine with a more efficacious hydrazide by using the MAOI hydrazide product to replace the toxic hydrazine which eliminates the slow metabolic conversion process. Such improvement would also increase efficacy over the hydrazine precursor which is being produced under product names as hydrazine sulfate, Procarbazine, Cefazolin, Fluconazole, Furazolidone, Carbidopa, and others. The MAOI hydrazide provides an efficacious substitute for the toxic hydrazine and additionally provides the speed, safety, and efficacy of the MAOI hydrazide which is much greater than what the acetylhydrazide metabolite can provide.

### BRIEF DESCRIPTION OF SEVERAL VIEWS OF THE DRAWING

(Not applicable)

### DETAILED DESCRIPTION OF THE INVENTION

Process of Making the Invention

This invention uses the existing hydrazide type mono amine oxidase inhibitor (MAOI) type antidepressant drug as a protein inhibitor for malignant disease purposes which constitutes a new purpose for the MAOI hydrazide use, and additionally provides improvements over existing hydrazine and hydrazide drugs not of the MAOI hydrazide class. Such new purpose and improvements does not require a chemical synthesis before the invention can be used.

Distinguishing this Invention from Others

Isoniazid and Iproniazid were the first hydrazide drug products discovered. Isoniazide has an open hydrazine terminal that provides the same type toxic effects as hydrazine yet was prefered as an antibiotic effective against tuberculosis by Fox in 1952. Fox had also tested Iproniazid as a potential antibiotic effective against tuberculosis but found no evidence the drug killed the tuberculosis organism. In 1953, Thompson discovered Marboran that provided a third hydrazide drug discovery that had a total spectrum antiviral drug capability but was based on a toxic laboratory chemical reagent that was not remedied and the product was discontinued. No other hydrazides have been found listed for medical purposes that the applicant can find which needs to be distinguished from the present invention. Additionally this invention is not about a new hydrazide drug composition but about a new medical purpose provided by the existing MAOI hydrazide drugs as provisioned by Kirby, 40 USPQ 368, and the improvement such MAOI hydrazides provide over the Marboran hydrazide and the hydrazine class of drugs. Hydrazine drugs are only metabolic precursors of the hydrazide prodrug principal that is improved upon by replacing the toxic hydrazine with the more efficacious, rapid acting, and cytotoxic free MAOI hydrazide drug. And a final class of selective protease enzymes inhibitor drugs exist that have particular molecular structures that windows protease cleavage targeting to only a specific protease enzyme for purposes to alter disease pathology as a treatments means. Such selective protease inhibitors are not relate to the present invention by mechanism, use, or purpose. No other types of hydrazide drugs are known to exist by the applicant.

MAOI Hydrazide Manufacture's Recommendations

The MAOI hydrazide drugs of the 1950's era which the present invention uses to shutdown cells that host a malignant disease, and a related antibiotic use, should not be used concurrently with other types of drugs that act on the CNS, or anything that has a hepatotoxic nature which includes alcohol. The same dosage levels, drug interaction precautions, and treatment regimen required for the MAOI hydrazide antidepressant type drugs should be followed for any new disease treatment purposes for which a hydrazide drug is used. The drug most used in this research effort was Iproniazid (Marsilid), that has a therapeutic dose of 50-150 mg daily. This drug was introduced about 1952 and has a safe history of medical uses for over 50 years as an antidepressant drug and is still used by some countries abroad (Drugs Available Abroad, Derwent Publications Ltd., page 250). Such hydrazide drugs are of the prodrug class and are not toxic per se as they are not chemically or medically active until cell protease cleavage targets the hydrazide substrate which then shuts down protein biosynthesis capability for the affected cells. However protein biosynthesis is necessary to provide enzyme systems needed to metabolize and detoxify other drugs and any toxic material exposure during therapy increases potential for complications. For that reason CNS acting drugs and drugs having hepatotoxic potential should be avoided to reduce chances for complications. A list of some such contradicted drugs are CNS depressants, narcotic analgesics, anticholinergics, and dibenzazepine antidepressants (Cutting's Handbook of Pharmacology, 6th ed., p. 628-629, 1979).

Best Modes for Using this Invention:

The principal concepts for using this invention is essentially the same as that for using the MAOI hydrazide pharmaceutical as an antidepressant drug. As such the same basic drug protocol, precautionary concerns, dosage regimen as is common when the drug is used for antidepressant type purposes also applies when used for any therapeutic purposes described herein. A variety of therapeutic applications were reduced to practice as pertaining to impromptu occasions when illness befell the applicant. However before any treatment concept was formulated or undertaken, a high-level of predictability for achieving the required results was evidenced by similar or analogous uses having been successfully performed by prior art inventions or experimental evidence. For example the antiviral drug Marboran of 1953, illustrates a serendipitous discovery based on a hydrazide substrate mechanism which shutdown smallpox, polio, and other viruses but the product had cytotoxic problems which were overcome by using the MAOI hydrazide for supplying the same antiviral capability but without the cytotoxic effects provided by Marboran. A second example pertains to Malazide of 1949, which was used to shutdown protein responsible for cell division and growth and did so in a benign and gentle way that the MAOI hydrazide readily provides to stop cancer cell division and growth and was evidenced safe for human uses based on mouse fetus experiments. And the antibiotic concept of this present invention has also been illustrated by the in vitro type prior art applications used to render live cells and bacteria sterile and static to preserve tissue homogenate from damage that provides evidence of a viable antibiotic mechanism for human use.

Such MAOI hydrazide drug use has been proven safe by a 50 year history of use as an antidepressant drug and has a very high level of predictability as a prodrug irreversible substrate protease inhibitor which can be separated into three areas of medical use illustrated by: (1) cells that host viral infections and are incessantly active producing viral metastatic proteins and related products; (2) cells which host cancer or other malignant disease where metastatic or aberrant protein products are produced; and (3) cells that are innate to infectious organisms that are active providing proteins that are toxic, pain producing, and sometimes dangerous in addition to incessant cell division, growth, and proliferation type protein requirements that are normal for most infectious organism. And the best mode for providing such medical use was based on the MAOI hydrazide pharmaceutical as is provided under the name of Ipronazid, as supplied by Aldrich Chemical of Milwaukee, catalog no.1-1,265-4. Iproniazid has been used for several different applications by prior art and provides a prodrug pharmaceutical mechanism used as an irreversible substrate protease inhibitor. And the best mode contemplated for remedial disease use in performance of this invention was provided by using the same protocol and dosage regimen listed by the manufacture for Iproniazid as used for antidepressant drug purposes. The dose level for Iproniazid is 50 to 150 mg daily which is sufficient to shutdown cell protein biosynthesis that inhibit oxidase protein production that yields the antidepressant mode of use, and is therefore sufficient to shutdown and inhibit metastatic and malignant proteins to provided a disease targeting and eradication mode of use.

Best Mode Contemplated to Treat Viral Infections

The RNA viral infection as HIV represents essentially begins when the retroviral genome is invaginated into a receptive host cell where the RNA is converted to DNA by reverse transcription. Then the DNA code becomes incorporated into the host cell DNA library. When the new DNA viral algorithm is activated it takes control of the host cell to provide a replication process that continues in recursive fashion to replicate the viral genome, which are the infectious metastatic packets which is a process that cannot stop and continues until the cell or essential components are destroyed. The viral process is dependent on essential components as protein and peptide products as the viral coat protein, peptides, maturation protein, various enzymes as RNA replicase subunit, and as many additional host cell protein products illustrate. As such protein production is an essential part of viral disease propagation and symptoms and any means used to disrupt or inhibit the protein production disrupts and inhibits viral disease activity.

As such a number of protease inhibitor drugs have been designed that will interfere with, or slow down the protein biosynthesis for temporary periods, but none have been designed around the MAOI type irreversible hydrazide substrate method that shuts down total protein biosynthesis activity in the cells that host such infection. Such process is illustrated by the MAOI hydrazide drug that supplies a hydrazide substrate which is an irreversible substrate protease inhibitor. The MAOI hydrazide supplies a substrate that is targeted by protease cleavage action during the incessant or higher level of protein biosynthesis activity ongoing in such host cells. However when protease cleavage targets the hydrazide the hydrazine radical is set free where it immediately bonds to the protease enzyme which obstructs protease and therefore shuts down protein biosynthesis activity in such host cells. Such action renders the host cell sterile and static that also renders the host cells to apoptosis where a replacement with a new cell that is disease free is expected to occur. The method is very rapid acting and applicable for remedial treatment use for all types of viral infections, whereas other types of protease inhibitors are disease specific, or uses reversible substrate mechanisms, or uses other short term obstructive means which only slows viral disease activity but which cannot terminate the host cell or stop the disease activity permanently as the hydrazide process provides.

Such remedial use based on the MAOI hydrazide drug Iproniazid, was reduced to practice when used to shutdown an HIV infection acquired by the applicant. This illness developed following an eye injury where the applicant received emergency room treatment and was in effect inoculated with HIV while in the emergency room. Such inoculation was followed within 3 or 4 days with a light fever and malaise, and within weeks with a persistent case of viral conjunctivitis that within about 18 months included persistent yeast infections, increasing malaise, arthritic pains, mental fatigue and many less significant symptoms. The condition did not improve or worsen substantially for a lengthy period thereafter until a purple discoloration on the front of the lower legs began to appear which is indicative of Kaposi's Sarcoma. Following the realization that full blown AIDS was next in order the applicant provided a blood sample for testing and was informed that he should seek treatment. Since there is no cure for such disease the applicant returned to the research interest he had participated in years earlier believing that Iponiazid had ended a bout with influenza whereas current treatment methods had little to offer the applicant as a cure. The applicant prepared to conduct and test a number of treatment related ideas as described in the Provisional Application 60/459,694, and began with a dose of about 100 mg of Iproniazid phosphate to determine what noticeable effect a single dose would provide. However such initial dose somewhat unexpectedly shutdown the viral activity seemingly in total as was indicated by the disappearance of the inflamation of the conjunctiva, improved energy, and relief of joint pain and such like. However after several weeks the virus begin to return as evidenced by the gradual reappearance of viral conjunctivitis and other symptoms and the planned research was then undertaken. After that study was completed the virus was eradicated in total where all symptoms and viral infections abated quickly and did not return.

Such rapid antiviral action and extreme antiviral efficacy has only been evidenced by one other invention which was also based on a the hydrazide mechanism as used by the present invention and was manufactured under the name of Marboran. And although the Marboran hydrazide evidenced amazing antiviral efficacy and rapid shutdown of the most virulent of viruses known as smallpox, polio, and other such viruses tested, Marboran was nevertheless made from a toxic laboratory chemical reagent which retained the toxic effects of the chemical reagent when compounded as a drug and was discontinued. In hindsight of the working hypothesis and the understanding it provides, it is clear that the Marboran cytotoxic problem could have been overcome in 1953 using a nontoxic MAOI hydrazide source. However the present invention now exceeds Marboran as a more efficacious antiviral agent without cytotoxic effects which is evidenced in this case as used to shutdown the HIV virus, viral conjunctivitis, and influenza. But most importantly Iproniazid has no cytotoxic effects which is evidenced by more that 50 years of safe use abroad as an antidepressant drug ((Drugs Available Abroad, Derwent Publications Ltd., page 250). As such the present invention based on the MAOI hydrazide constitutes a major improvement over prior art Marboran as a total spectrum antiviral agent. The best mode contemplated to shutdown such viral disease is to administer a pharmaceutical preparation of an MAOI hydrazide drug at a dose level as MAOI use would require, or less, until the associated viral disease symptoms have disappeared and do not return.

Best Mode Contemplated to Treat Cancer

The cancer disease model as envisioned by the applicant that provides a similar process to the viral infection example which serves to further illustrate a disease model addressed by the hydrazide process. Cancerous cells are incessantly active protein producing cells that supply protein products for cell division and growth. Cells that host cancer must also supply the metastatic and protein supported RNA packets which is in essence the cancer algorithm or genome. Such RNA packet with its tough protein compliment while still intact are continually replicated and pushed out of the cancer host cell which can then become invaginated by adjacent or nearby receptive cells to provide metastasis of the cancer mechanism except in some aggressive cancers where the metastatic packet may find a receptive host cell in other organs not so close by. Once the metastatic packet is invaginated by a receptive host cell then reverse transcription occurs where the RNA cancer encoded program is converted back into DNA similar to the action of the RNA virus where it then becomes part of the DNA host cell library. When the newly installed DNA cancer code is activated the cancer program takes control of the cell which replicate the metastatic packet or cancer genome without ending such activity. Such malignant disease activity makes tremendous demand on protein resources that renders cancer cells very vulnerable to the hydrazide mechanism. This is because substrate resources are in great demand if not totally depleted most of the time in the cells that host a malignant disease mechanism such that the hydrazide substrate is without proteolytic substrate competition and is therefore immediately targeted by protease cleavage when introduced into the cell. Protease cleavage of the hydrazide substrate then transfers the hydrrazine radical moiety to the protease enzyme where it bonds irreversibly to provide the irreversible substrate mechanism that halts all protein biosynthesis capability which renders the cancer host cell sterile and static. Within a window of about two weeks aopoptosis and cell replacement occurs where a new cell that is free of the cancer is provided.

The MAOI hydrazide anticancer concept was reduced to practice by the applicant when an impromptu opportunity appeared after a decade old mole began to metastasize. The growth was about a half inch in diameter located in the center of the applicant's forehead, and had remained stable, symmetrical, and unchanged for years when it began to increase in size, shape, and having mixed pigmentation indicative of malignant melanoma. At such time the applicant began a treatment regimen based on Iproniazid Phosphate at about 100 mg daily for about three or four weeks when the perimeter of the growth began to separate from the skin surface underneath and itch. The applicant would unconsciously pick at the protruding edge as if picking at a scab which served to more quickly separate the neoplastic part at its perimeter where sections were peeled away. Over a period of days the cancer like neoplasm was removed in total which exposed the epidermal layer that soon took on a normal appearance without any signs of cancerous tissue remaining.

A second more benign cancer growth coexisted at the same time while the melanoma treatment was ongoing which was of the basal cell carcinoma type which was not very noticeable, bothersome, or believed to be life threatening, but which also disappeared following the hydrazide melanoma treatment. The basal cell carcinoma was located below the left eye on the applicant's cheek which was characterized by a hard, gritty, cellular formation where scratching or rubbing the surface would dislodge some of the small hard cells which is a common characteristic of basal cell carcinoma. Although the condition was not apparent or at a life threatening stage at the time, and had never raised concerns or the idea of providing opportunity for Iproniazid screening as the melanoma cancer did, it was nevertheless inadvertently shutdown by Iproniazid and had likewise disappeared. Such successful use of Iproniazid as a cancer treatment method constitutes a new cancer treatment use for the MAOI hydrazide product. The best mode contemplated to purge cells from the system which provides host to a cancer or other malignant mechanism is to administer a pharmaceutical preparation of an MAOI hydrazide drug at a dose level as MAOI use would require, or less, until the associated manifestations and symptoms have disappeared and do not return.

Best Mode Contemplated to Treat Multiple Myeloma

The applicant provides a concept for purposes to help visualize the molecular level process of multiple myeloma mechanism in context of the MAOI hydrazide drug action that the applicant believes will rapidly terminate the disease. The applicant believes that multiple myeloma is based on a mechanism that began when a section of DNA is damaged which has the genetic instructions for a protein product required by bone marrow. The mechanism likely has a genesis based on damages that occur to the DNA following free radical, radiation, or toxic substance type damages which are the kinds of damages likely to accumulate with age. A systemic need for such protein would activate the DNA instruction code program that would provide the proper protein biosynthesis action up to such location where the damage existed which would provide an error or void which alters the required protein product which is then unusable. The defective protein thus provided cannot satisfy the systemic need that initiated the DNA program and therefore successive signals communicate the need for such protein that keeps the cell active providing the defective or malignant protein product. Such aberrant and damaging protein product produces pain and damages to bone, kidneys, and other organs, and the protein product while still attached to the RNA is metastatic and pished out of the cell. Such metastatic packet could then become invaginated into adjacent cells to further metastasize the myeloma action that in effect began as damage to a single nucleotide in one cell that had become corrupted by free radical, radiation, or toxic substance type damage to the nucleotide.

The incessant disease activated protease system producing such aberrant myeloma protein products would quickly target the hydrazide irreversible substrate that would quickly shutdown protein biosynthesis to provide immediate pain relief and comfort to the patient without causing any untoward side effects. Secondly the disease would be placed in full remission very quickly due to hydrazide substrate prodrug action that would shutdown protein biosynthesis activity in all cells that host the myeloma algorithm which would then follow with cell apoptosis and replacement with new cells as provided by cell division from a healthy cell. Such myeloma remedial action could easily be verified using animal models based on the MAOI drug Iproniazid as supplied by Aldrich Chemical as is commonly used to preserve tissue homogenates. The mode contemplated to purge myeloma cells from the system is to administer the MAOI hydrazide antidepressant drug at a dose level as MAOI antidepressant use would require until the aberrant protein emissions cease and the disease is in full remission.

Best Mode Contemplated to Treat Parkinson's Disease

The applicant provides a hypothetical concept for purposes to help visualize the molecular level process of Parkinson's disease in context of MAOI hydrazide action on the host cells. The applicant believes that Parkinson's disease has a genesis common with other malignant protein diseases where a specific product is needed as can be provided by instructions encoded in a DNA sequence but because such DNA code has been damaged at some nucleotide by free radical, radiation, or toxic substance type action the product produce by such DNA instruction is systemically worthless, damaging, and metastatic. Because the systemic need that activated the DNA program can not be satisfied by the defective product then repetitive DNA activations requesting such protein product occurs. Successive requests can only be responded to by producing the same defective product which in this case may be the DNA instruction code or algorithm needed to synthesize dopamine as is the likely product absent from the basal ganglia that generates Parkinson's disease symptoms. This DNA damage hypothesis is also supported by fact that known causes of Parkinson's have been traced to many different DNA damaging events such as exposure to anti-psychotic drugs, encephalitis letharaga virus, carbon monoxide poisoning, cerebrovascular disease, and use of drugs synthesized in error having irreversible damage potential to the basal ganglia.

In this case the damage is likely to have occurred in a DNA code instruction sequence that provides for the decarboxylase enzyme protein instructions where the resultant enzyme is defective and the dopamine product normally produced retains the carboxyl radical which cannot be used, and cannot satisfy cell needs for decarboxylation of the dopamine precursor. Such defective DNA instruction code when activated by a systemic needs would produce the defective product which would be metastatic before the RNA is separated and therefore some metastatic packets would be pushed out of the cell to become invaginated into nearby cells that would gradually metastasize and worsen the disease. Therefore any means to inhibit such protein product would constitute a beneficial disease treatment method and also slow progression of the disease. Such hypothesis is supported by fact that protein inhibitor drugs constitute the principal treatment means for Parkinson's disease. In effect the most successful treatment of Parkinson's disease has only been accomplished by medications having protein inhibiting properties which could be better provided or improved upon using a MAOI hydrazide drug that not only inhibits the malignant protein product, but renders the corrupted cells that host such disease sterile and static where the offending cells would be terminated by apoptosis and be replaced by a disease free healthy cell. Such method could be expected to provide a much more efficacious and rapid treatment means based on the hydrazide irreversible substrate process. The mode contemplated by the applicant to purge such DNA damaged cells responsible for Parkinson's disease from the system is to administer a pharmaceutical preparation of an MAOI hydrazide drug at a dose level as MAOI use would require, or less, until the associated symptoms have disappeared and do not return, or until no further improvements are recognized to occur.

Best Mode Contemplated to Treat Biological Regulation Disorders

The applicant provides a hypothetical concept for purposes to help visualize the molecular level process of a corrupted DNA algorithm sequence as results from free radical, physical, radiation, or toxic substance type damages that accumulate with age to negate the allosteric properties required for biological communication schemes. Such schemes are use in biological communications processes where molecular binding to a specific site, or where peptide hormonal scheme must likewise match a specific receptor site to activate some function or communicate some need. Such biological communications are prevented when the DNA instructions used to synthesize such allosteric product are altered in any small way. The applicant believes that the MAOI hydrazide when used over a period of time can restore such faulty DNA code by apoptotically terminating the cell at fault. Such biological communication schemes are used by polypeptides, hormone, prostaglandin product, enzyme or like protein or peptide products which all have a unique spelling and allosteric properties that corresponds only to a complimentary matching allosteric interface which communicates some systemic need or status. Any corruption of any aspect of such property prevents the communication processes from occurring which the applicant believes is largely responsible for age related problems. Treatment to restore the proper spelling sequence and allosteric properties is possible in a limited respect in that the hydrazide drug mechanism is able to shutdown protease and force cell replacement by means of the irreversible substrate action where the DNA damaged cell will in time be forced to undergo apoptosis and be replaced with a new cell derived from a healthy cell absent of such DNA damage. Accordingly such replacement would have the correct spelling and allosteric program in its DNA code which has not been damaged and thus provides means for an identical copy to be provided by cell division as needed to restore the proper cell function.

Because such DNA damages accumulate with age the applicant believes that such accumulated damage is a principal manifestation of ageing. Adult diabetes provides an illustrative example based on the applicant's theory where a general diminishing of insulin levels are seen with age as would result from free radical, radiation, or toxic substance type damages to the DNA instruction code as pertains to the insulin allosteric and spelling properties. Such damage could begin where a single error alters an encoded segment into the long spelling sequence of the insulin molecule which in most cases negates totally or very severely diminishes the insulin properties provided by the altered product. Such damage would require more and more insulin producing cells to remain active longer to produce enough viable insulin molecule to satisfy the systemic needs until a deficit develops that requires medical intervention to provide the additionally needed insulin. The insulin molecule damage scenario is only one of very many where corruption of the allosteric or spelling properties or a protein sequence or hormone product negates or prevents the biological communications in any one of its many forms.

The applicant believes the effects of such aberrant peptide or protein products are manifested in numerous ways illustrated in part as one possible cause for allergic, metabolic, endocrine, neurologic, arthritic, obesity, schizophrenia, autoimmune, anorexia, colitis, pain sensitivity, hypertension, psoriasis, cravings, addictions, eating disorders, and countless additional possibilities that could easily be determined based on positive results that would occur after a long trial period of hydrazide substrate therapy. Hydrazide administered over a lengthy period of time would slowly purge such aberrant or abnormal protein producing cells which would then be replaced with new cells having full DNA integrity that are disease and damage free. The speed at which such age related malady could be reversed would correspond to how frequently the cell is activated to produce its intended protein product where protease cleavage would be invoked. The incessantly active cells providing a corrupted protein biosynthesis product would be more immediately purged using the hydrazide method whereas the less active cells that are seldom involved in protein biosynthesis would take a more lengthy period for effective treatment to result. The best mode contemplated to purge and replace such DNA damaged cells responsible for biological communications, hormonal deficiencies is to administer a pharmaceutical preparation of an MAOI hydrazide drug at a dose level as MAOI use would require, or less, and for a lengthy period of time until no further improvements occur.

Best Mode Contemplated for Antibiotic Use

The hydrazide drug prevents peptide productions that controls cell division, growth, and proliferation of cells innate to infectious organisms as bacterial, fungal, protozoal, metazoal, and others. Such mechanism provides a new antibiotic purpose to shutdown the cells innate to infectious organisms that are continually providing protein for cell division, growth, and toxic protein generation. Hydrazide has not been recognized as an antibiotic method probably because conventional antibiotics kill infectious organisms after several days which is evidenced by the apoptotic fragments seen by microscopic assay which would suggest to researchers that hydrazides have no antibiotic value because the organisms do not appear to be killed. However the value of antibiotics is not how quickly it kills the infectious organism but how quickly it can render a dangerous infectious organism sterile, static, and harmless such that the organism cannot multiply or produce toxic or dangerous proteins which is provided much faster by rendering the organisms sterile and static by MAOI hydrazide action than any conventional antibiotics can kill them. It is the toxic and dangerous pain producing or venomous protein products that makes the comparative difference between a benign acidophilus bacterium and the extremely dangerous anthrax type. However within minutes the rapidly replicating and toxin producing anthrax organisms would be rendered sterile, static, and harmless by the hydrazide method whereas more than four or five days may be required when using conventional antibiotic methods before results begin to appear. Such new antibiotic purpose provides a total spectrum antibiotic whereas other antibiotics have a limited spectrums of use peculiar to the type of antibiotic used. This invention was first reduced to practice by this applicant followed a meal comprising leftovers that was followed with extreme stomach pain and griping, indicative of bacterial gastroenteritis. The condition was quickly remedied after taking a 100 mg dose of Iproniazid with a glass of warm tap water. Within about 5 minutes it was evident that the concept was extremely efficacious and rapid acting as most pain was gone and within about 10 minutes all pain and griping had disappeared and did not return.

As such hydrazides may not be impressive to kill infectious organisms as a conventional antibiotics assay would reveal but it is impressive for purposes to more quickly render such infectious organisms sterile, static, and harmless. And if for some reason it would be desired to quickly kill off such sterile and static infectious organisms sooner than otherwise would happen, then the hydrazide method can be used concurrently with conventional antibiotics having no hepatotoxic nature as an adjuvant drug. Such combined use with a compatible antibiotic would increase synergy and benefits of both antibiotic methods and thus kill the infectious organisms faster. Such combination would also provide an adjuvant purpose which would hold infectious organism reproduction and proliferation in check and in essence allow much smaller amounts of the products to be used to provide the same antibiotic result. In such case where the MAOI type hydrazide drug is concurrently used, the infectious organism would be incapable of developing and passing on antibiotic resistance traits to successive generations. Antibiotic resistant organisms have become a major problem due to world population levels that are increasingly dependent on a limited number of antibiotics. Such problems are also exacerbated by the unnecessary and frivolous use of antibiotics by the livestock industry, and such problems caused by abuse of conventional antibiotics would be negated if a MAOI hydrazide drug were prescribed concurrent with compatible antibiotic preparations or added to animal feed at minimum levels as a prophylaxis drug. The successful use of the MAOI as a general purpose type antibiotic drug provides a new purpose for the MAOI hydrazide drug. The mode contemplated to render infectious organisms sterile, static, and benign is to administer a pharmaceutical preparation of an MAOI hydrazide drug at a dose level as MAOI antidepressant use would require, or less, over a period of time until the associated symptoms of an infectious organism has disappeared or when no further improvements are recognized to occur.

Summation

Although the level of predictability in the art of medicine is considered to be relatively low, the hydrazide irreversible substrate mechanism is well defined, evidenced, and very predictable and is somewhat illustrated by fact all MAOI hydrazide drugs provides the same mechanisms and purposes and are interchangeable in their applications, use and purposes they serve. This high level of predictability is because this invention pertains to a hydrazide prodrug mechanism where the predictability of the irreversible substrate prodrug action is certain and can only exist in one of two states, either cell protein biosynthesis is of a sufficient level indicative of a malignant disease mechanism that invokes the irreversible substrate shutdown of cell protein biosynthesis, or else nothing happens to the cell at all. Such predictability is illustrated by all prior art cited, and in particular where cell division and growth was first inhibited in plants by Malazide hydrazide; then Iproniazid hydrazide was used to inhibit the tuberculosis organism reproduction and growth; then successfully predicted to likewise inhibit cell division and growth in mice fetus experiments; then successfully inhibited cell division and growth of microorganisms as a homogenate preservative, which the applicant then predicted would inhibit infectious organisms in vivo to provide a new antibiotic purpose and mechanism successful used to shutdown bacterial gastroenteritis. Then smallpox, polio, and other viruses were discovered to be inhibited by Marboran hydrazide which the applicant predicted could be inhibited better by any one of the MAOI hydrazides drugs and successfully used Iproniazid to inhibit the HIV/AIDS virus and influenza. And because hydrazines are also transformed into hydrazides by metabolism in vivo as an effective anticancer drug then the applicant predicts that the MAOI will better perform such cancer therupy and used Iproniazid to inhibit and shutdown melanoma cancer, basal cell carcinoma, and a Kaposi's Sarcoma. In summation the claims set fourth by this application are well founded, evidenced, and have successfully been reduced to practice repeatedly with phenomenal results to inhibit and shutdown the influenza virus, then the HIV virus, also viral conjunctivitis, and melanoma cancer, also basal cell carcinoma, a Kaposi's Sarcoma cancer, and was finally use to inhibit and shutdown a case of bacterial gastroenteritis. Such successful applied use has reduced to practice the invention in accordance with Corona v. Dovan, 273 US 692, 1928 CD 252, and the successful use of the MAOI hydrazide drug for malignant disease purposes which constitutes a new medical purpose for the MAOI hydrazide class re Kirby, 40 USPQ 368.

The applicant believes this invention is of very special importance at this time of war, predictions of mutant bird flu and crown virus outbreaks, and the ever increasing HIV pandemic. Such concerns have been addressed under MPEP 708.02, first because this invention provides a major HIV/AIDS drug breakthrough provisioned for under 708.02(x). An estimated 50 million people have HIV/AIDS and a new victim dies every few minutes that the present MAOI drug can easily stop from happening when made available for public use, or when distributed to the needy by humanitarian organizations. And secondly MPEP 708.02(x), provides privilege pertaining to cancer which is a disease that robs practically every family of beloved members which this invention can prevent when made available for public use which justifies the accelerated processing of this application. And a third basis for MPEP 708.02(xi) privilege pertains to countering terrorism which this invention provides by fact it provides the best defense possible against anthrax and viral related WMD infections, and mutant bird flu or crown virus outbreaks. Such outbreak is deemed most likely to occur in a third world location that will unfortunately empower individual terrorist the opportunity for martyrdom by sacrificing their own lives as a means to spread the infection here before symptoms become obvious. A successful WMD attack against our nation would require that it occur before a national defense capability as the MAOI hydrazide drug provides, is made available for public use which is why MPEP 708.02(xi) provides expedited processing which makes available sooner such inventions which also minimizes time available for foul play, delay, or improvised obstructions that may foil processing, approval, or availability of such life saving drugs for what ever motive, whether foreign or domestic, or motivated by personal gain or religious motives.

And finally the applicant requests help in drafting patent claims as provisioned for under MPEP 707.07(j) be provided if the claims drafted by the applicant are deemed insufficient or can otherwise be improved upon by the examiner. The pro se applicant is not familiar with the proper preparation and prosecution of the patent application, has been unable to find a patent attorney not already under drug company contracts, or willing to represent a case where medical knowledge and experience are deemed important. And because time is crucial the applicant has no choice but to stop looking for an attorney and prosecute the case himself and therefore makes request pursuant to MPEP 707.07(j) for help as needed that the examiner can provide, and any direction the examiner can provide the applicant to avoid any one of the many pitfalls possible for the novice is also requested and very much appreciated if provided.

The applicant claims the following:

1. The mono amine oxidase inhibitor hydrazide drugs provides an irreversible substrate process that targets and shuts down protein production in cells that have a high level of protein biosynthesis activity as exists in active CNS cells producing oxidase and other protein products which renders said cells sterile and static that provides the original antidepressant drug purpose, wherein a new purpose comprises use of said drugs and process to target and render sterile and static cells with a level of biosynthesis indicative of a malignant disease activity whereby cells that host viral infections, cancer and malignant or metastatic disease mechanisms are targeted and eradicated.

2. The mono amine oxidase inhibitor hydrazide drugs provides an irreversible substrate process that targets and shuts down protein production in cells that have a high level of protein biosynthesis activity as exists in active CNS cells producing oxidase and other protein products which renders said cells sterile and static that provides the original antidepressant drug purpose, wherein a new purpose comprises use of said drugs and process to target and render sterile and static the cells innate to infectious organisms which provides a new antibiotic purpose whereby said cells are unable to provide cell division, proliferation, or biosynthesis of toxic or damaging protein products.

3. The isatin thiosemicarbazone type hydrazide drugs provide an irreversible substrate process that targets and shuts down protein biosynthesis in cells with a high level of protein biosynthesis activity as exists in cells that host a viral infection which renders said cells sterile and static and doomed for apoptosis, but said process retains the cytotoxic property provided by the isatin thiosemicarbazone type hydrazide; wherein the improvement comprises replacing said cytotoxic isatin thiosemicarbazone type hydrazide with a mono amine oxidase inhibitor type hydrazide that provides said process without cytotoxic effects whereby improved antiviral capability with increased efficacy are provided.

4. The hydrazine drugs are transformed by metabolism into the hydrazide active principal that provides the irreversible substrate process that targets cells having protein biosynthesis activity indicative of cells that host a malignant disease mechanism, however hydrazine is toxic and said metabolism process is slow and incomplete; wherein the improvement comprises using a mono amine oxidase inhibitor hydrazide drug to replace the toxic hydrazine drug precursor which provides the same benefits and scope of medical uses as provided by the hydrazine but with improvements in speed, efficacy, and without the toxic effects of hydrazine.

5. A method for targeting and rendering sterile and static cells with a high level of ongoing protein biosynthesis activity by supplying said cells with hydrazide.

6. A method for the treatment of diseases caused by cells that host a malignant disease mechanism is provided by administering to a patient in need thereof an effective amount of a mono amine oxidase inhibitor hydrazide drug which then shuts down the ongoing protein biosynthesis in said cells which renders said cells sterile, and static, which induces apoptosis whereby said cells are replaced with new cells that are free of said disease.
